(19) United States
(12) Patent Application Publication
(10) Pub. No.: US 2026/0355014 A1
(43) Pub. Date: Nov. 12, 2026

(54) ADJUSTABLE SHUNTING SYSTEMS AND METHODS OF MANUFACTURING THE SAME

(71) Applicant: Shifamed Holdings, LLC, Campbell, CA (US)

(72) Inventors: Tom Saul, Portland, OR (US); Sateeshchandra Bajikar, San Jose, CA (US)

(21) Appl. No.: 19/127,893

(22) PCT Filed: Nov. 17, 2023
(86) PCT No.: PCT/US2023/080290

§ 371 Date: May 7, 2025

Related U.S. Application Data
(60) Provisional application No. 63/426,632, filed on Nov. 18, 2022.
(60) Provisional application No. 63/476,676, filed on Dec. 22, 2022.

(51) Int. Cl. A61F 9/007

(52) U.S. Cl. CPC A61F 9/00781

(57) ABSTRACT

The present technology is generally directed to adjustable shunting systems for controlling fluid flow between a first body region and a second body region. The shunting systems can include multiple components that can be separately manufactured and coupled together to form each individual system. For example, the systems can include an actuator assembly, a control assembly, and a fluidics assembly.

### CROSS-REFERENCE TO RELATED APPLICATION(S)

This application claims priority to U.S. Provisional Patent Application No. 63/426,632, filed Nov. 18, 2022, and U.S. Provisional Patent Application No. 63/476,676, filed Dec. 22, 2022, both of which are incorporated herein by reference in their entireties.

### TECHNICAL FIELD

The present technology generally relates to implantable medical devices and, in particular, to adjustable shunting systems for promoting fluid flow between a first body region and a second body region of a patient.

### BACKGROUND

Implantable shunting systems are widely used to treat a variety of patient conditions by shunting fluid from a first body region/cavity to a second body region/cavity. For example, shunting systems have been proposed for treating glaucoma. The flow of fluid through the shunting systems is primarily controlled by the pressure gradient across the shunt and the physical characteristics of the flow path defined through the shunt (e.g., the resistance of the shunt lumen). Conventional, early shunting systems (sometimes referred to as minimally invasive glaucoma shunts or “MIGS”) have shown clinical benefit; however, there is a need for improved shunting systems and techniques for addressing elevated intraocular pressure and risks associated with glaucoma, as well as other patient conditions. For example, there is a need for shunting systems capable of adjusting the therapy provided, including the flow rate/fluid resistance between the two fluidly connected bodies. As another example, there is a need for a shunting system capable of being modified after manufacture (e.g., in the clinic) to personalize the system for the patient and/or as part of the clinician's plan for the implant procedure.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1A illustrates an adjustable shunting system configured in accordance with select embodiments of the present technology.

FIG. 1B is an exploded view of the adjustable shunting system of FIG. 1A.

FIG. 1C is a cross-sectional view of the adjustable shunting system of FIG. 1A.

FIG. 2 is a partially schematic illustration of select portions of the adjustable shunting system of FIGS. 1A-1C.

FIG. 3 is another partially schematic illustration of select portions of the adjustable shunting system of FIGS. 1A-1C.

FIG. 4 is another partially schematic illustration of select portions of the adjustable shunting system of FIGS. 1A-1C.

FIGS. 5A and 5B illustrate an adjustable shunting system configured in accordance with another embodiment of the present technology.

FIGS. 6A-6C illustrate another adjustable shunting system configured in accordance with select embodiments of the present technology.

FIGS. 7A-7C illustrate additional features of an actuator assembly of the adjustable shunting system of FIGS. 6A-6C.

FIGS. 8A and 8B illustrate another adjustable shunting system configured in accordance with select embodiments of the present technology.

FIGS. 9A and 9B illustrate another adjustable shunting system configured in accordance with select embodiments of the present technology.

FIG. 10 illustrates a cap assembly configured for use with the adjustable shunting systems of FIGS. 8A-9B and configured in accordance with select embodiments of the present technology.

FIG. 11 illustrates a microfluidic channel extending through a plate assembly of the system of FIGS. 9A and 9B and configured in accordance with select embodiments of the present technology.

### DETAILED DESCRIPTION

The present technology is generally directed to adjustable shunting systems for controlling fluid flow between a first body region and a second body region. The disclosed shunting systems can include multiple components that can be separately manufactured and coupled together to form each system. For example, the systems can include an actuator assembly, a control assembly, and a fluidics assembly. As described in detail below, the systems can utilize manufacturing techniques that enable the systems to be produced with suitable precision and in bulk.

In some embodiments, the present technology provides an optically actuated, latching or non-latching, fluid microvalve capable of varying fluid flow rate and/or fluid resistance through it at any given input pressure between a low value and a high value. The ratio of the high to low flow rates and/or resistances (e.g., “open” and “closed” states) can be set by design by varying the dimensions of the appropriate elements (as described in greater detail below) and may be in excess of about 10:1, or about 8:1, or about 6:1. The valve is suitable for use with liquids and gasses, such as to control the flow of fluid through an adjustable shunting system. The microvalve design is suitable for scaling to ≤mm X≤few mm X≤10s mm in overall dimensions.

In some embodiments, the shunting systems and/or microvalves described herein are suitable for being manufactured in parallel at the wafer and/or die assembly scale using a combination of various MEMS fabrication processes and techniques with combinations of selected materials. The design is compatible with material and process choices for the microvalve to be biocompatible and suitable for long-term implantation in human subjects/animals when used in conjunction with an appropriate housing.

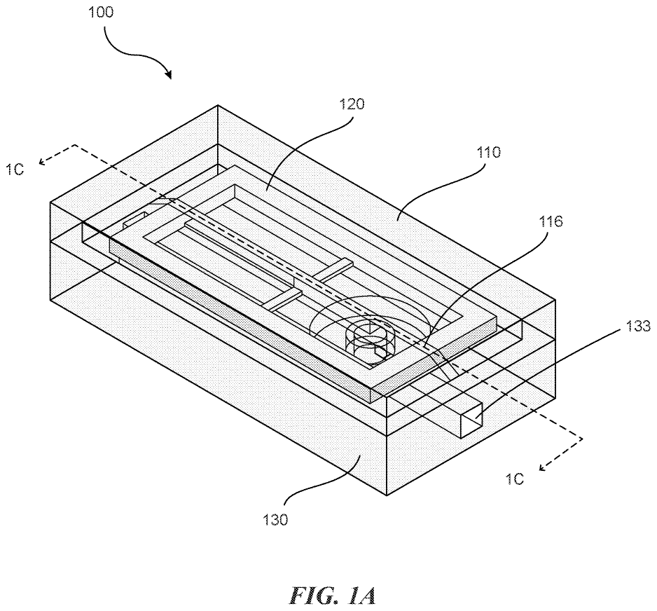

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples and claims but are not described in detail with respect to FIGS. 1A-11.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the use of relative terminology, such as “about,” “approximately,” “substantially,” and the like, refers to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

Reference throughout this specification to the term “resistance” refers to fluid resistance unless the context clearly dictates otherwise. The terms “drainage rate” and “flow rate” are used interchangeably to describe the movement of fluid through a structure at a particular volumetric rate. The term “flow” is used herein to refer to the motion of fluid, in general.

The systems described herein can be designed for shunting fluid between a variety of body regions. For example, many of the embodiments described herein are designed to be implanted in a patient's eye to shunt aqueous between the anterior chamber and a target outflow location (e.g., a subconjunctival bleb space), such as to treat glaucoma. However, although certain embodiments are described in terms of shunting fluid from an anterior chamber of an eye, one of skill in the art will appreciate that the present technology can be readily adapted to shunt fluid from and/or between other portions of the eye or, more generally, from and/or between a first body region and a second, different body region of a patient. Moreover, while the certain embodiments herein are described in the context of glaucoma treatment, any of the embodiments herein, including those referred to as “glaucoma shunts” or “glaucoma devices,” may nevertheless be used and/or modified to treat other diseases or conditions, including other diseases or conditions of the eye or other body regions. For example, the systems described herein can be used to treat diseases characterized by increased pressure and/or fluid buildup, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

FIGS. 1A-1C illustrate an adjustable shunting system 100 (“the system 100”) configured in accordance with select embodiments of the present technology. More specifically, FIG. 1A is a perspective view of the system 100, FIG. 1B is an exploded view of the system 100, and FIG. 1C is a cross-sectional view of the system 100 taken along the line 1C-1C indicated in FIG. 1A. As described in greater detail below, the system 100 is configured to provide a titratable therapy for shunting fluid from a first body region to a second body region, such as shunting aqueous from an anterior chamber of a patient's eye to a target outflow location.

Referring to FIGS. 1A and 1B together, the system 100 includes at least three distinct components that can be separately manufactured and assembled to form the system 100: (a) an actuator component or assembly 110, (b) a control component or assembly 120, and (c) a fluidics component or assembly 130. As best shown in FIG. 1B, the actuator assembly 110 can include a cover or lid 111 having a top surface 112 and a plurality of side surfaces 113. The cover 111 can be composed of glass, silicon, or another generally rigid material. The actuator assembly 110 can further include a trim layer, flange, or perimeter 114 extending around an interior perimeter of the side surfaces 113 of the cover 111. An actuator 116 can extend across the actuator assembly 110 from a first portion of the trim layer 114 to a second, opposite portion of the trim layer 114. In some embodiments, the trim layer 114 and the actuator 116 comprise a unitary component (e.g., manufactured using additive or subtractive manufacturing techniques as single component). In some embodiments, the trim layer 114 and the actuator 116 are at least partially composed of a shape memory material, such as Nitinol or Nitinol-containing materials. In some embodiments, the Nitinol has a transition temperature between a first material state (e.g., martensitic, R-state, etc.) and a second material state (e.g., R-state, austenitic) greater than body temperature. As a result, the shape memory properties of the actuator 116 can be utilized to drive actuation of the system 100, as described below. The actuator assembly 110 also can include a cavity or recess 118 facing away from the top surface 112 (e.g., facing downward in the illustrated configuration).

As also best shown in FIG. 1B, the control assembly 120 includes a frame 122 and a flow control element 124. The frame 122 is configured to fit at least partially within the cavity 118 of the actuator assembly 110. The flow control element 124 is configured to be controlled by the actuator 116 to control fluid flow through the system 100, as described below. In some embodiments, the control assembly 120 includes one or more support elements 126 (shown as a T-shaped beam although other configurations are possible) configured to support the flow control element 124 and/or provide a pivot point for the flow control element 124. The control assembly 120 can be composed of silicon or another suitable material.

The fluidics assembly 130 defines a lumen or channel 132 for transporting fluid between a first body region and a second body region of a patient when the system 100 is implanted in the patient. The lumen 132 includes an inlet 133, an outlet 134, and a flow restrictor portion 136. The fluidics assembly 130 also includes a thinned portion or diaphragm 138 covering the flow restrictor portion 136. As described below, the diaphragm 138 is at least partially deflectable/deformable into the flow restrictor portion 136 to selectively control fluid resistance through the lumen 132. In some embodiments, the fluidics assembly 130, including the diaphragm 138, is composed of glass, silicon, silicone, or other suitable materials.

In an assembled configuration (e.g., shown in FIG. 1A), the actuator assembly 110, the control assembly 120, and the fluidics assembly 130 are vertically stacked and coupled together (e.g., chemically bonded, glued, etc.). As set forth above, the control assembly 120 sits at least partially within the cavity 118 defined by the actuator assembly 110. The inherent interference between the control assembly 120 and the actuator 116 (when assembled together as shown in FIG. 1A) therefore induces an upward bow or otherwise deforms/deflects the actuator 116 upwardly toward the top surface 112 of the actuator assembly 110. This deflection induces strain in the actuator 116, priming it for actuation, as described below. Also in the assembled configuration, the flow control element 124 is positioned between the actuator 116 and the diaphragm 138. In some embodiments, the flow control element 124 is coupled/affixed to the actuator 116. As a result of this configuration, the actuator 116 sits within a sealed chamber 140 (FIG. 1C) between an upper surface of the fluidics assembly 130 and a lower surface of the actuator assembly 110. In some embodiments, an inert gas (e.g., argon) can be disposed within the sealed chamber 140. Without intending to be bound by theory, it is expected that filling the sealed chamber 140 with argon or another inert gas (rather than having the sealed chamber be a vacuum) may obviate issues associated with preloading the diaphragm 138 via the pressure differential associated with the vacuum. It is further expected that filling the sealed chamber 140 with argon or another inert gas (or having the sealed chamber 140 under vacuum) may improve the heat transfer properties associated with the actuator 116 as compared with having the features of the system 100 in an aqueous environment (such as in the patient's eye).

When the system 100 is implanted between a first body region and a second body region of a patient, and as best shown in FIG. 1C, fluid flows through the lumen 132 between the inlet 133 and the outlet 134 (labeled as a “fluidics path” in FIG. 1C). The fluid passes through the flow restrictor portion 136, which includes flowing up and over a projection or extension 137, which in the illustrated embodiment includes a raised annular lip. A user can selectively control the flow of fluid through the lumen 132 generally, and the flow restrictor portion 136 specifically, by actuating the actuator 116. In particular, actuating (e.g., heating above a transition temperature) a first portion 116a of the actuator 116 causes the first portion 116a of the actuator 116 to contract, which pivots the flow control element 124 in a clockwise direction relative to the view shown in FIG. 1C toward a first (e.g., closed) position. In turn, the flow control element 124 pushes into the diaphragm 138 and deflects the diaphragm 138 downwardly. This decreases a dimension of the flow restrictor portion 136 through which fluid can pass, thus increasing a fluid resistance through the flow restrictor portion 136. Indeed, in some embodiments, actuating the first portion 116a of the actuator 116 pushes the diaphragm 138 downward to an extent that the diaphragm 138 forms a substantial or even complete seal with the projection 137, effectively stopping fluid from flowing through the lumen 132. The operation can be reversed by actuating (e.g., heating above a transition temperature) a second portion 116b of the actuator 116, which causes the second portion 116b to contract, thereby pivoting the flow control element 124 in a counterclockwise direction relative to the view shown in FIG. 1C toward a second (e.g., open) position. In turn, the flow control element 124 disengages from (or at least ceases to press downwardly on) the diaphragm 138. This increases a dimension of the flow restrictor portion 136 through which fluid can pass, thus decreasing a fluid resistance through the flow restrictor portion 136. The flow control element 124 can therefore be selectively and repeatedly transitioned between at least two different positions (e.g., the first position and the second position) that impart a different fluid resistance through the flow restrictor portion 136 and therefore, for a given pressure differential, a different flow rate through the lumen 132.

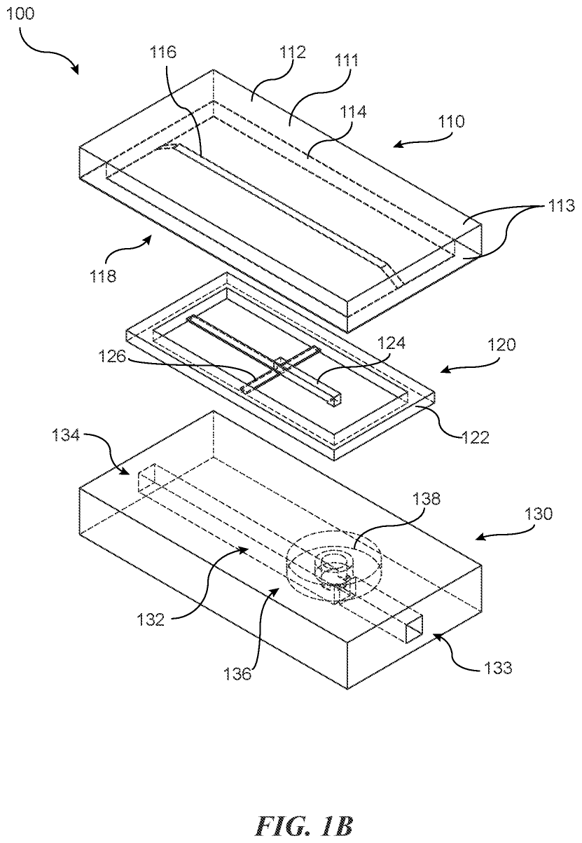

FIG. 2 illustrates a portion of the system 100 including one of the side surfaces 113 of the actuator assembly 110 (FIG. 1B), the actuator 116, the flow control element 124, one of the support elements 126, and the diaphragm 138, with other aspects of the system 100 omitted for purposes of clarity. More specifically, FIG. 2 illustrates a displacement/deformation (in microns) of each of the foregoing components when the flow control element 124 is in the first (e.g., closed) position. As shown, the flow control element 124 and the diaphragm 138 are each displaced when the flow control element 124 occupies the first (e.g., closed) position.

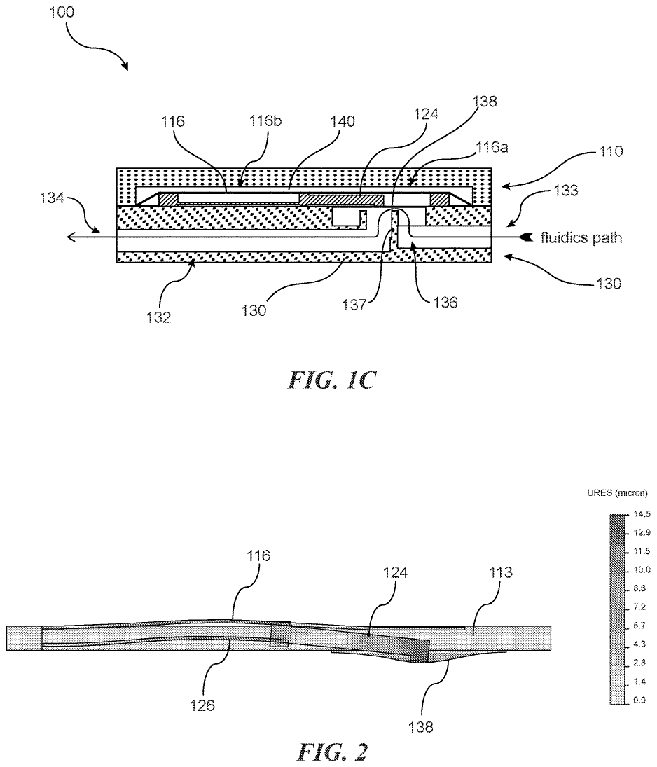

FIG. 3 illustrates the actuator 116, the control assembly 120, and the diaphragm 138, with other aspects of the system 100 omitted for purposes of clarity. More specifically, FIG. 3 illustrates a displacement/deformation (in microns) of each of the foregoing components when the flow control element 124 is in the first (e.g., closed) position. As shown, the actuator 116 is deformed by virtue of the control assembly 120 pushing upwardly on the actuator 116 during assembly of the system 100, described previously.

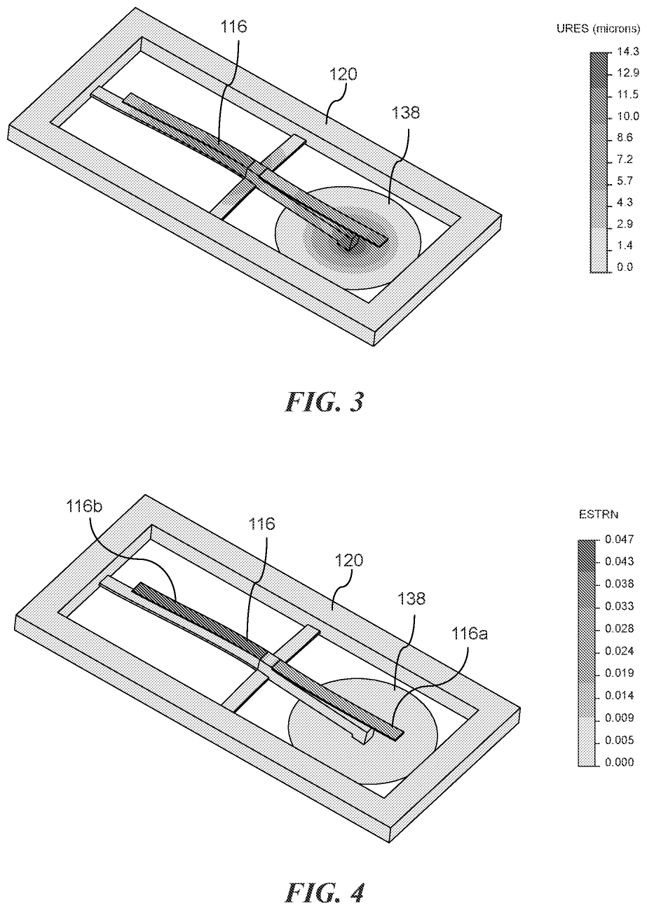

FIG. 4 also illustrates the actuator 116, the control assembly 120, and the diaphragm 138, with other aspects of the system 100 omitted for purposes of clarity. FIG. 4 illustrates strain induced in the actuator 116 following actuation of the first portion 116a of the actuator 116 (e.g., to drive the flow control element 124 toward the first position). As shown, there is more strain in the second portion 116b than the first portion 116a following actuation of the first portion 116a. This is because actuating the first portion 116a causes the first portion 116a to transition to a different material state (e.g., austenitic) and toward a default/shape memory (e.g., manufactured, shape-set, etc.) geometry. In contrast, actuating the second portion 116b would reduce the strain in the second portion 116b and increase the strain in the first portion 116a. Accordingly, the first portion 116a and the second portion 116b of the actuator 116 generally work in opposition, enabling repeated and selective actuation between the first position and the second position. Additional details regarding, and examples of, bi-directional shape memory actuators that can be used with the present technology are described in U.S. Patent Application Publication Nos. 2020/0229982 and 2021/0251806, the disclosures of which are incorporated by reference herein in their entireties and for all purposes.

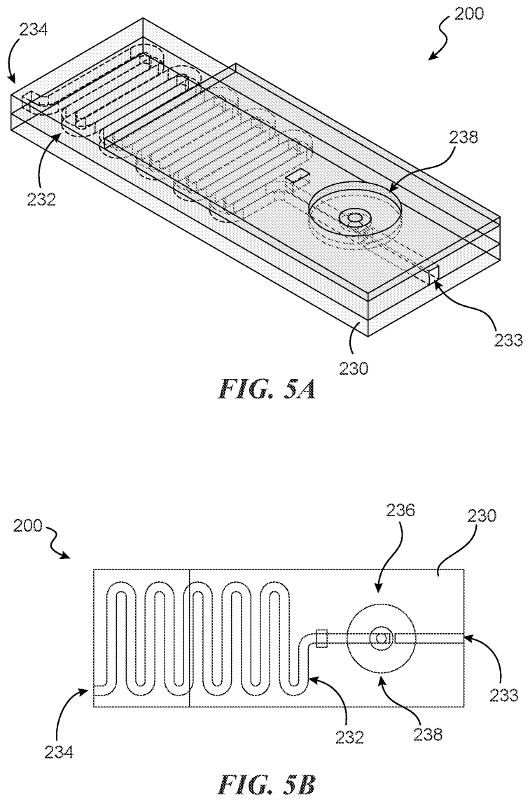

FIGS. 5A and 5B illustrate a portion of an adjustable shunting system (“the system 200”) configured in accordance with another embodiment of the present technology. More specifically, FIG. 5A is a perspective view of a fluidics component or assembly 230 of the system 200 and FIG. 5B is a top view of the fluidics assembly 230. The illustrated fluidics assembly 230 includes a number of features identical or generally similar to certain features of the system 100 described above with reference to FIGS. 1A-1C. For example, the fluidics assembly 230 is configured to be assembled with an actuator component or assembly (e.g., the actuator assembly 110—FIG. 1B) and a control component or assembly (e.g., the control assembly 120—FIG. 1B) to form a complete system 200. In other embodiments, however, the fluidics assembly 230 may be assembled with other suitable actuator and/or control components to form an adjustable shunting system.

The fluidics assembly 230 includes a lumen or channel 232 for transporting fluid between a first body region and a second body region of a patient when the assembled system 200 is implanted in the patient. The lumen 232 has an inlet 233 at a first side of the fluidics assembly 230 and an outlet 234 at a second, opposite side of the fluidics assembly 230. The lumen 232, however, differs from the lumen 132 of the fluidics assembly 130 described above with reference to FIGS. 1A-1C in that a portion of the lumen 232 has a serpentine arrangement (rather than a generally straight/linear arrangement like the lumen 132). The lumen 232 accordingly has a greater length and a higher resistance (e.g., a fluid or flow resistance) than the lumen 132.

The fluidics assembly 230 further includes a flow restrictor portion 236 (FIG. 5B) along the lumen 232 between the inlet 233 and the outlet 234. The fluidics assembly 230 also includes a thinned portion or diaphragm 238 covering the flow restrictor portion 236. As described previously, the diaphragm 238 is at least partially deflectable/deformable into the flow restrictor portion 236 to selectively control fluid resistance through the lumen 232. The fluidics assembly 230, including the diaphragm 238, may be composed of glass, silicon, silicone, or other suitable materials.

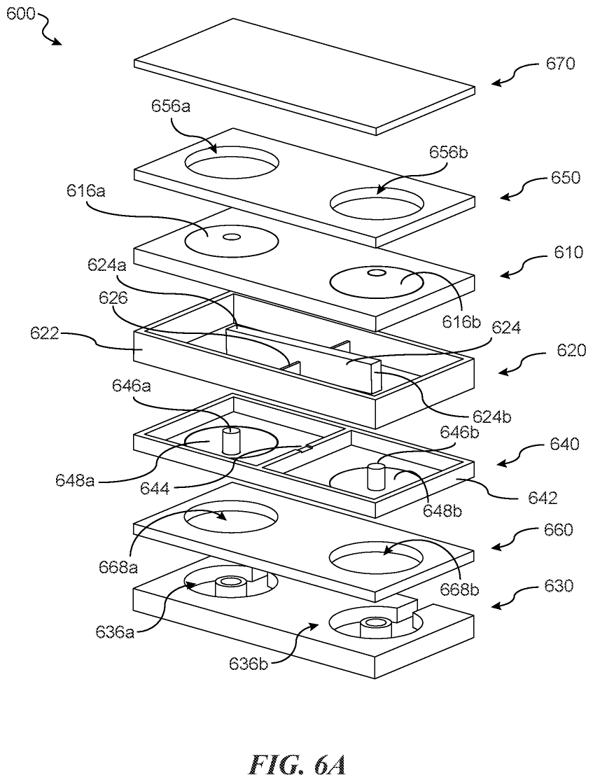

FIGS. 6A-6C illustrate a portion of an adjustable shunting system (“the system 600”) configured in accordance with another embodiment of the present technology. More specifically, FIG. 6A is an exploded view of the system 600, FIG. 6B is a top view of the system 600, and FIG. 6C is a cross-sectional view of the system 600 taken along the line 6C-6C shown in FIG. 6B. As described in greater detail below, and similar to the systems described above, the system 600 can be composed of a plurality of discrete pieces, components, or layers that can be separately manufactured and then coupled together to form the system 600.

As shown in FIG. 6A, the system 600 includes an actuator component or assembly 610, a first control component or assembly 620, a second control component or assembly 640, and a fluidics component or assembly 630. The system 600 can further include a first spacer 650, a second spacer 660, and a cover or lid 670. The various components can be stacked and coupled (e.g., adhered) together to form an assembled configuration, such as the configurations shown in FIGS. 6B and 6C.

Referring collectively to FIGS. 6A-6C, the actuator assembly 610 can include a first actuator 616a and a second actuator 616b. Similar to the actuator 116 described with respect to FIGS. 1A-4, the first actuator 616a and the second actuator 616b can be composed of a shape memory material (e.g., Nitinol) having a phase transformation temperature greater than body temperature. However, relative to the actuator 116 of the system 100, the first actuator 616a and the second actuator 616b can have a relatively thin and circular or disc-like shape such that the first actuator 616a and the second actuator 616b form a diaphragm-like element. In other embodiments, the first actuator 616a and/or the second actuator 616b can have other suitable shapes in which its width and length are about the same, e.g., rectangular (e.g., square) shaped, pentagonal shaped, hexagonal shaped, etc. As described in greater detail below, the first actuator 616a and the second actuator 616b can be selectively actuated (e.g., heated) to change one or more flow characteristics (e.g., fluid resistance) through the system 600. As best shown in FIG. 6C, a first actuator standoff or pin 617a can extend downwardly from the first actuator 616a and a second actuator standoff or pin 617b can extend downwardly from the second actuator 616b. As described in greater detail below, the first actuator pin 617a and the second actuator pin 617b are configured to transfer motion of the first actuator 616a and the second actuator 616b, respectively, to the first control assembly 620 to change one or more fluid characteristics (e.g., fluid resistance, flow rate, etc.) of the system 600.

The first control assembly 620 can be generally similar to the control assembly 120 described with reference to FIGS. 1A-4. For example, as best shown in FIG. 6A, the first control assembly 620 can include a frame 622 and a movable control element 624 (e.g., an arm, a pendulum, a level, etc.). The movable control element 624 can extend between a first end portion 624a and a second end portion 624b. When the system 600 is in an assembled configuration, and as best shown in FIG. 6C, the first end portion 624a of the movable control element 624 is configured to align with the first actuator pin 617a of the actuator assembly 610, and the second end portion 624b of the movable control element 624 is configured to align with the second actuator pin 617b of the actuator assembly 610. As shown in FIG. 6A, the first control assembly 620 can further include a support structure 626 extending between parallel walls of the frame 622 to provide support to the movable control element 624. The first control assembly 620 can be composed of silicon, glass, Nitinol, or other suitable materials.

The second control assembly 640 can include a frame 642 and a support structure 644 extending between parallel walls of the frame 642. When the system 600 is in an assembled configuration, and as best shown in FIG. 6C, an underside of the movable control element 624 can rest on or sit at least partially within a groove or notch in the support structure 644. In some embodiments, the support structure 644 supports a central portion 624c of the movable control element 624 between the first end portion 624a and the second end portion 624b, and can therefore act as a fulcrum for rotation of the movable control element 624, described in greater detail below. Referring to FIGS. 6A and 6C together, the second control assembly 640 further includes a first flow control diaphragm 648a and a second flow control diaphragm 648b. A first standoff or connecting element 646a extends upward from the first flow control diaphragm 648a toward an underside of the movable control element 624, and a second standoff or connecting element 646b extends upward from the second flow control diaphragm 648b toward an underside of the movable control element 624. The first connecting element 646a is configured to transfer motion from the movable control element 624 to the first flow control diaphragm 648a, and the second connecting element 646b is configured to transfer motion from the movable control element 624 to the second flow control diaphragm 648b. The second control assembly 640 can also be composed of silicon, silicone, glass, Nitinol, or other suitable materials.

As best shown in FIG. 6C (when the system 600 is in an assembled configuration), the actuator assembly 610, the first control assembly 620, and the second control assembly 640 define a chamber 680 (e.g., a hermetically sealed chamber). As described in greater detail below, the movable control element 624 can be configured to rotate within the chamber 680 in response to actuation of the first actuator 616a and/or the second actuator 616b. The chamber 680 can be a vacuum or can be filled with a gas such as, in some embodiments, an inert gas (e.g., argon) which, as described previously, is expected to improve certain operational properties of the systems relying upon shape memory actuation.

When the system 600 is in an assembled configuration, the first flow control diaphragm 648a and the second flow control diaphragm 648b of the second control assembly 640 sit over a first flow restrictor portion 636a and a second flow restrictor portion 636b, respectively, of the fluidics assembly 630. In some embodiments, the first flow restrictor portion 636a and/or the second flow restrictor portion 636b can be generally similar to the flow restrictor portion 136 of the system 100 (FIGS. 1A-1C). For example, the first flow restrictor portion 636a and the second flow restrictor portion 636b can each include a raised annular lip extending upward toward an underside of the respective flow control diaphragm, with a fluid path extending through the annular lip. Thus, movement of the first flow control diaphragm 648a and/or the second flow control diaphragm 648b can change a flow characteristic (e.g., a fluid resistance) through the fluidics assembly 630, similar to the description above with reference to FIGS. 1A-4. In some embodiments, the first flow restrictor portion 636a and the second flow restrictor portion 636b form part of a common fluid pathway through the system 600. In other embodiments, the first flow restrictor portion 636a and the second flow restrictor portion 636b form different fluid pathways through the system 600. The fluidics assembly 630 can be composed of silicon, silicone, glass, Nitinol, or other suitable materials.

Referring to FIGS. 6A and 6C together, the first spacer 650 is configured to sit at least partially between the actuator assembly 610 and the cover 670, and includes (a) a first actuator aperture 656a for receiving, and accommodating movement of, the first actuator 616a, and (b) a second actuator aperture 656b for receiving, and accommodating movement of, the second actuator 616b. In some embodiments, the empty space defined by the first actuator aperture 656a and/or the second actuator aperture 656b can be a hermetically sealed chamber (by virtue of the cover 670 being hermetically sealed to the first spacer 650, which can be hermetically sealed to the actuator assembly 610). In such embodiments, the empty space of the first actuator aperture 656a and/or the second actuator aperture 656b can be a vacuum or filled with an inert gas such as argon.

The second spacer 660 is configured to sit at least partially between the second control assembly 640 and the fluidics assembly 630, and includes (a) a first diaphragm aperture 668a for receiving, and accommodating movement of, the first flow control diaphragm 648a, and (b) a second diaphragm aperture 668b for receiving, and accommodating movement of, the second flow control diaphragm 648b. The first spacer 650 and the second spacer 660 can be composed of silicon, glass, Nitinol, or other suitable materials. The cover 670 can be composed of glass or other transparent material that permits the first actuator 616a and the second actuator 616b to be selectively actuated (e.g., via laser actuation as described throughout).

In operation, the system 600 enables a user to selectively change a flow characteristic (e.g., fluid resistance) through the fluidics assembly 630 by virtue of changing the degree of deflection of the first flow control diaphragm 648a and/or the second flow control diaphragm 648b. For example, the first flow control diaphragm 648a can be movable between at least a first (e.g., closed) position in which it restricts, plugs, or even seals fluid from flowing through the first flow restrictor portion 636a of the fluidics assembly 630, and a second (e.g., open) position in which it permits more flow through the first flow restrictor portion 636a. The second flow control diaphragm 648b can similarly be movable between first and second positions providing different fluid resistances through the second flow restrictor portion 636b. For purposes of illustration, FIG. 6C illustrates the first flow control diaphragm 648a in the first (e.g., closed) position and the second flow control diaphragm 648b in the second (e.g., open) position. Thus, in the configuration shown in FIG. 6C, fluid may be blocked from flowing through the first flow restrictor portion 636a but may be permitted to flow through the second flow restrictor portion 636b.

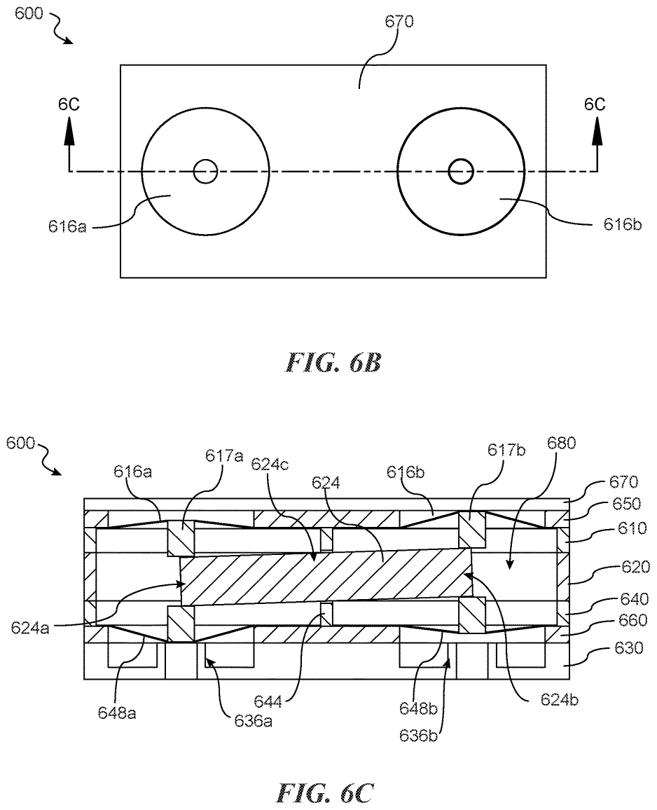

The first flow control diaphragm 648a and the second flow control diaphragm 648b can be moved (e.g., between at least the first and second positions) via actuation of the first actuator 616a and the second actuator 616b, respectively, and corresponding motion of the movable control element 624. For example, FIG. 7A illustrates the actuator assembly 610 in a pre-assembled configuration before it has been loaded into the system 600, with other aspects of the system 600 omitted for purposes of illustration. As shown, in a pre-assembled configuration, the first actuator 616a and the second actuator 616b are relatively flat (the term “relatively flat” may have different meanings depending on the application-in some cases, the term “relatively flat” refers to a component lying within the same general plane or layer; in other cases, the term “relatively flat” refers to the respective components being significantly wider than they are tall, or vice versa). This represents the default/shape memory geometry (e.g., manufactured geometry, shape-set geometry, heat-set geometry, etc.) of the first actuator 616a and the second actuator 616b.

FIG. 7B illustrates the actuator assembly 610 after it has been loaded into the system 600 but before it has been actuated. As shown, loading the actuator assembly 610 deforms (e.g., pre-strains) both the first actuator 616a and the second actuator 616b, causing them both to flex, bow, tent, or otherwise deform upwardly (e.g., into the corresponding first actuator aperture 656a and the second actuator aperture 656b shown in FIG. 6A). This is because as the system 600 is assembled, the first actuator pin 617a and the second actuator pin 617b engage the movable control element 624, which applies a generally similar upward force on both the first actuator pin 617a and the second actuator pin 617b.

FIG. 7C illustrates the actuator assembly 610 after the first actuator 616a has been actuated (e.g., heated above a transition temperature to transform from a first material state having relatively less stiff mechanical properties to a second material state having relatively stiffer mechanical properties). As shown, actuating the first actuator 616a causes the first actuator 616a to move toward its default geometry, which as set forth above is generally flat within a single plane (FIG. 7A). As the first actuator 616a moves toward its default geometry, the first actuator pin 617a moves downwardly and applies a greater downward force on the first end portion 624a of the movable control element 624 (FIGS. 6A and 6C) relative to any downward force the (unactuated) second actuator 616b and the second actuator pin 617b apply on the second end portion 624b of the movable control element 624. In some embodiments, a ratio between the downward forces applied by the first actuator pin 617a and the second actuator pin 617b is greater than about 1.5:1, greater than about 2:1, greater than about 3:1, greater than about 4:1, greater than about 10:1, and/or greater than about 20:1. The imbalance between the downward forces on either end of the movable control element 624 rotates the movable control element 624 in a counterclockwise direction and pushes the first flow control diaphragm 648a (FIGS. 6A and 6C) downwardly into the first flow restrictor portion 636a via the first connecting element 646a. Depending on the magnitude of the downward force applied, the first flow control diaphragm 648a can seal or substantially seal the first flow restrictor portion 636a and prevent or substantially prevent fluid from flowing therethrough. Because the movable control element 624 rotates about its central portion 624c, the second end portion 624b rotates upwardly as the first end portion 624a rotates downwardly. This further deforms the second actuator 616b relative to its default geometry (which remains in the first material state having relatively less stiff mechanical properties) by pushing the second actuator pin 617b upwardly, or otherwise permitting the second actuator pin 617b to deflect upwardly. The operation can be reversed by actuating the second actuator 616b, which causes clockwise rotation of the movable control element 624 and pushes the second flow control diaphragm 648b into the second flow restrictor portion 636b. Accordingly, the actuator assembly 610 can be repeatedly and selectively actuated to change one or more fluid characteristics through the fluidics assembly 630.

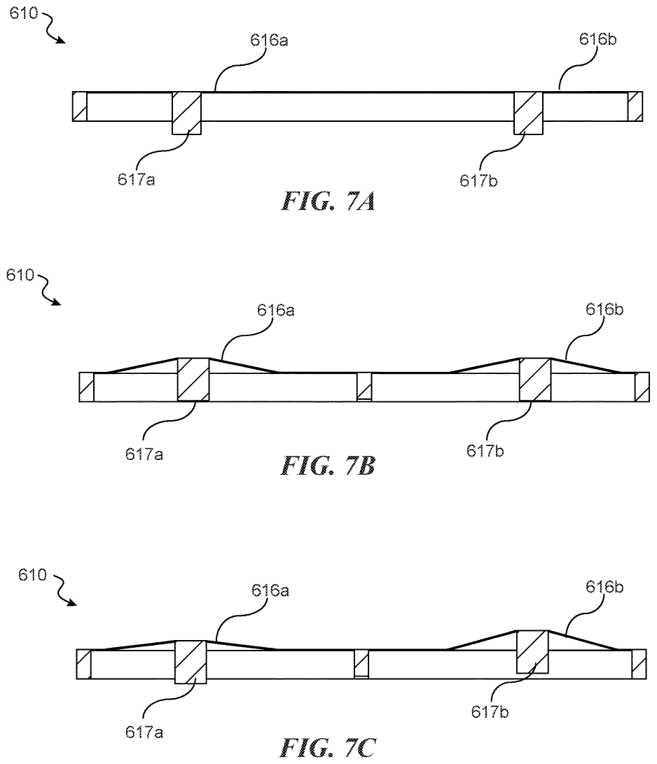

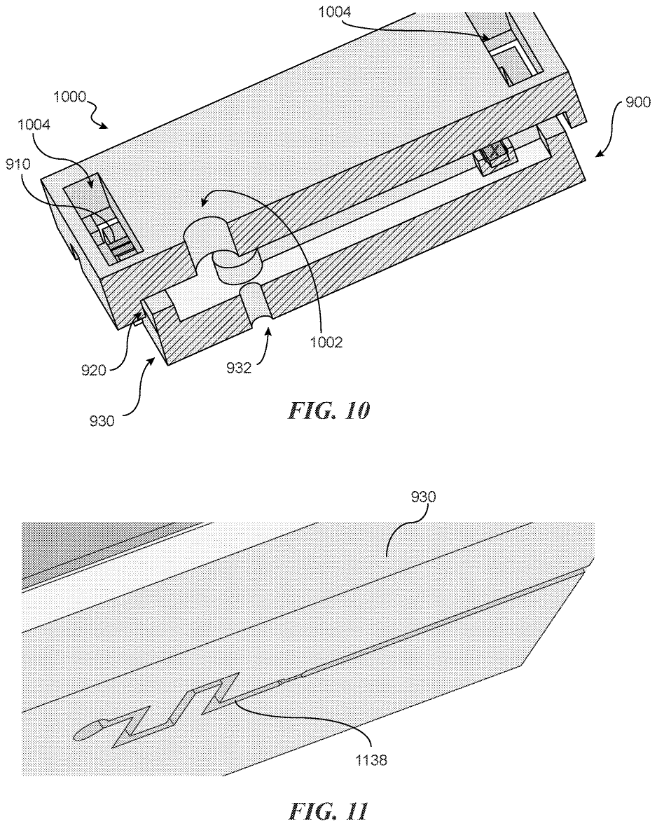

FIG. 8A illustrates another adjustable shunting system 800 (“the system 800”) configured in accordance with select embodiments of the present technology. The system 800 can include an actuator 810, a control or microvalve assembly 820, and a fluidics assembly or plate 830. Flow is regulated in the system 800 by selectively moving a control element 824 (which in the illustrated embodiment is a flat paddle plate and is integrally formed with the body of the control assembly 820) placed in proximity to the fluidics assembly 830. In some embodiments, a lower surface of the control element 824 is spaced apart from an upper surface of the fluidics assembly 830 by between about 0.1 and about 1.0 micron. In other embodiments, the lower surface of the control element 824 may slidably engage an upper surface of the fluidics assembly 830. Regardless, the control element 824 is positioned generally adjacent a hole or aperture 832 in the fluidics assembly 830. In some embodiments, the hole 832 has a diameter of between about 5 microns and about 50 microns, or a diameter that is at least smaller than a surface area of a lower surface of the control element 824 so that the control element 824 variably obscures the hole 832 over its range of motion, as described below. FIG. 8B, which is an enlarged cross-sectional view of the control element 824 and the hole 832, shows that the hole extends vertically through the fluidics assembly 830, e.g., such that it drains to a lumen or other flow pathway. In some embodiments, such as described in detail below with reference to FIG. 11, the fluidics assembly 830 can itself have one or more microfluidic channels extending at least partially therethrough and configured to route fluid to and/or from the hole 832.

The ratio of the various fluid resistances through the hole 832 that can be obtained by selectively manipulating a position of the control element 824 (and thus the ratio of the high to low flow rates that can be achieved through the hole 832) is determined in part by the diameter of the hole 832, the diameter of the control element 824, the fraction of the hole 832 that can be obscured in various positions of the control element 824, the vertical spacing between the control element 824 and the hole 832, and the configuration of the system 800 components itself (e.g., the configuration of mechanical suspension beams 826 and the actuator 810).

The control assembly 820 further includes a plurality of mechanical suspension or support beams or members 826 that are arranged in a cantilevered or clamped manner and integrated with a frame 822 of the control assembly 820. The mechanical suspension beams 826 can ensure that the control element 824 maintains a desired vertical spacing from the upper surface of the fluidics assembly 830 over the entire range of motion of the control element 824. In various embodiments, single or multiple mechanical suspension beams 826 may be used to control stiffness in different directions and guide motion of the control element 824 to the desired path and positions. The mechanical suspension beams 826 are designed to provide a relatively large vertical stiffness to lateral stiffness ratio so that the control element 824 may move substantially parallel to the upper surface of the fluidics assembly 830 while avoiding contact therewith.

Similar to the embodiments described throughout this Detailed Description, the control element 824 can be selectively transitioned between two or more positions providing two or more different fluid resistances through the hole 832 via actuation of the actuator 810. And as also similar to the various embodiments described herein, the actuator 810 can comprise a shape memory material (e.g., Nitinol) having a material phase transformation temperature greater than body temperature such that shape memory properties can be used to induce movement in the actuator 810, and thus induce movement of the control element 824. For example, the actuator 810 can include pre-strained patterned films or wires of Nitinol that are coupled to the control assembly 820. In some embodiments, the actuator 810 can be heated (and thus actuated) using an external laser or other optical energy source that is focused on a portion of the actuator 810. Selective heating of portions of the Nitinol actuator, that is appropriately designed, with the external focused laser enables controlling the directivity of the force/torque and therefore the direction of actuation.

In some embodiments, the Nitinol composition may be optionally selected to exhibit superelastic behavior at the operating temperature to achieve different behaviors. The use of operation-temperature superelastic Nitinol films to actuate the actuator 810 can enable latching or non-latching switching behavior between two or more positions depending on the design and arrangement of the mechanical suspension beams 826 and the composition and behavior of the actuator 810. For example, a bistable, double-clamped curved beam suspension, such as the mechanical suspension beams 826 shown in FIG. 8A, would enable latching behavior with potential positions of the control element 824 determined by the suspension design. As another example, a monostable double-clamped suspension or a cantilevered suspension in conjunction with an operation-temperature superelastic Nitinol actuator would enable non-latching valve behavior. The use of a shape memory alloy composition Nitinol actuator element will enable a latching behavior with a monostable simple double-clamped suspension or a cantilevered suspension with the control element positions determined largely by the hysteresis behavior of the Nitinol actuator.

In some embodiments, the system 800 can be fabricated in a combination of borosilicate glass and silicon. In particular, the fluidics assembly 830 can be made of glass and the control assembly 820 (e.g., the frame 822, the control element 824, and the mechanical suspension beams 826) can be made of silicon. The glass fluidics assembly 830 contains the hole 832, which can be wet/dry etched, laser drilled, or formed by molding the glass wafer using a silicon template wafer and a bonding, reflow, polishing wafer process. The silicon control assembly 820 can be formed using, e.g., a (Bosch) Deep Reactive Ion Etching process in a single crystal silicon wafer using an appropriate mask. Without intending to be bound by theory, this technique is expected to enable the production of a high aspect ratio mechanical suspension beam 826 with a high vertical stiffness to lateral stiffness ratio that is desirable for operation of the control assembly 820, as described above. In some embodiments, one or more cavities of varying target depths can be etched into the surface of the silicon forming the underside of the control assembly 820 that will contact the glass of the fluidics assembly 830 to avoid contact over these portions and allow the control element 824 to move freely parallel to the hole 832 and maintain the desired spacing of the control element 824 to the upper surface of the fluidics assembly 830 for the designed on/off flow ratio. Additionally or alternatively, these gaps/cavities forming a spacing between portions of the control assembly 820 and the fluidics assembly 830 may be patterned and/or etched into the surface of the glass (e.g., into the upper surface of the fluidics assembly 830). In some embodiments, the silicon can be coated with a thin, thermally grown oxide layer to promote biocompatibility. In some embodiments, the silicon wafer (e.g., the control assembly 820) is aligned and bonded to the glass wafer (e.g., the fluidics assembly 830) using anodic or fusion bonding.

In some embodiments, the fluidics assembly 830 can feature a patterned thin metal film, preferably of the same Nitinol or other shape memory material as the actuator 810, to allow selective optical transparency over portions of the fluidics assembly 830. Both the inner and outer sides of the fluidics assembly 830 may incorporate such patterned films.

The actuator 810, which as set forth above can be composed of Nitinol or another suitable shape memory material, can be deposited and patterned on a separate wafer and released from it, resulting in freestanding patterned foil. Integration of the actuator 810 into the control assembly 820 can be accomplished at the wafer or die level using pick and place microassembly tools. The actuator 810 can be attached (e.g., permanently attached) to the control assembly 820 by dispensing adhesive droplets (~few 10s um diameter) at targeted bonding locations.

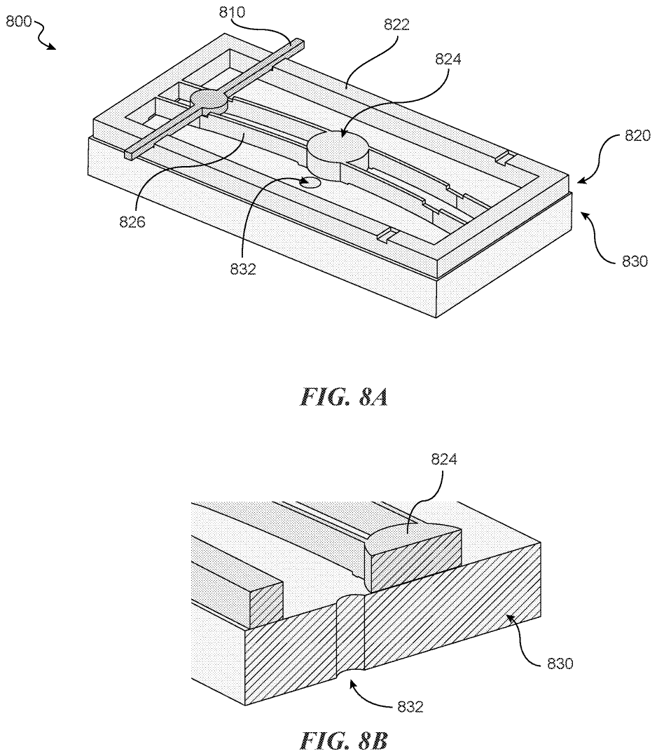

FIG. 9A illustrates another adjustable shunting system 900 (“the system 900”) configured in accordance with select embodiments of the present technology. The system 900 can be generally similar to the system 800 described with reference to FIGS. 8A and 8B. For example, the system 900 can include a fluidics assembly or plate 930 having a hole or aperture 932 extending therethrough (FIG. 9B). The system 900 further includes a control assembly 920 having a frame 922, a control element 924, and a mechanical suspension beam 926. In contrast to the system 800 described above with reference to FIGS. 8A and 8B, the system 900 includes a single mechanical suspension beam 926 that does not extend between opposite and parallel sides of the frame 922. That is, the control element 924 exists at a free or unconstrained end of the control assembly 920. The system 900 also includes an actuator 910, which can be a shape memory actuator having a material phase transformation temperature that is greater than body temperature such that its shape memory properties can be used to induce motion in the actuator 910, and thus the control assembly 920, similar to the description above. The actuator 910 can be secured to the control assembly 920 using adhesive droplets at target bonding locations, which are marked with an “X” in FIG. 9A. The system 900 can be manufactured using similar techniques as described above with reference to the system 800 of FIGS. 8A and 8B.

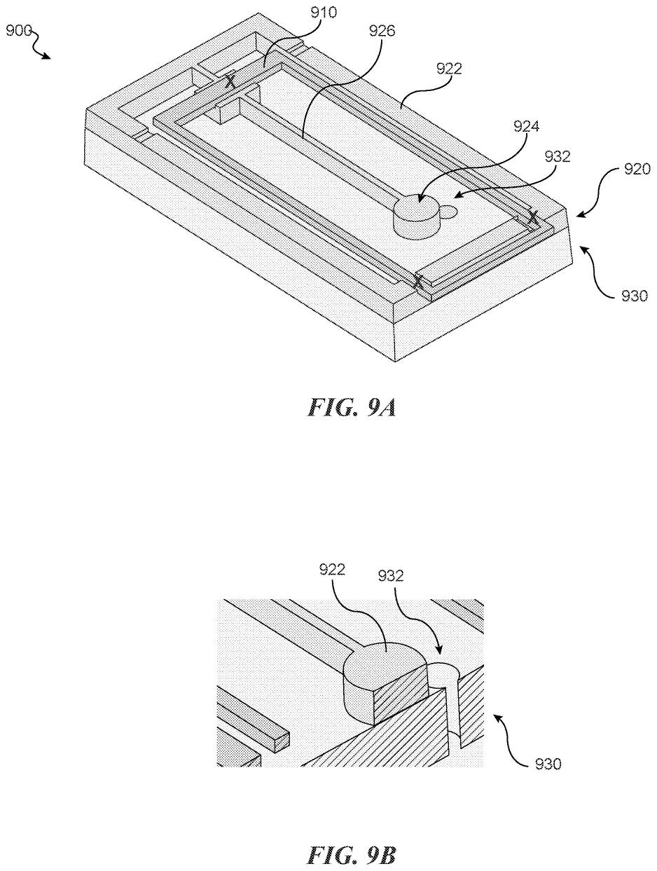

The system 800 described with reference to FIGS. 8A and 8B and the system 900 described with reference to FIGS. 9A and 9B can include additional features beyond those illustrated in FIGS. 8A-9B. For example, FIG. 10 illustrates a cap 1000 that is configured in accordance with select embodiments of the present technology and that can be used in connection with either the system 800 or the system 900. More specifically, FIG. 10 is a cross-sectional view of the cap 1000 extending over the system 900. Although shown with the system 900, the cap 1000 can equally be used with the system 800, as one skilled in the art will appreciate.

The cap 1000 can be used to close off the top face of the system 800 or the system 900. In some embodiments, the cap 1000 includes a fluid transfer hole 1002 for permitting fluid to flow through the cap 1000. The cap 1000 (or alternatively a special tooling wafer) may be designed and fabricated to engage with select portions of the actuator 910 that overhangs the control assembly 920, pushing the portions of the actuator 910 perpendicular to the plane occupied by the control assembly 920, prior to the completion of adhering all the attachment points of the actuator 910 to the control assembly 920. In some embodiments, only one portion of the actuator 910 is locked to the control assembly 920 using an adhesive prior to assembly of the cap 1000 over the actuator 910 (marked in FIG. 10 using an “X”). The engagement of the actuator 910 with the cap 1000 or assembly tool wafer during assembly motion may then be used to induce the desired pre-strain (e.g., deformation) to the actuator 910 based on the designed overlap of the cap 1000 and the control assembly 920. The pre-strained actuator 910 can then be locked into the control assembly 920 by completing the application of adhesive to the remaining attachment locations through adhesive application windows 1004 in the cap 1000. In some embodiments, the cap 1000 can be fabricated in a glass or silicon wafer using fabrication techniques similar to those described earlier.

In some embodiments, the fluidics assembly 830 or 930 may further include one or more fluidics channels that connect to the hole in the fluidics assembly and that can be used to provide a designed fluid resistance in series with the system 800 or 900. For example, FIG. 11 illustrates the fluidics assembly 930 of FIGS. 9A and 9B with a fluidics channel 1138 extending at least partially therethrough. In the embodiment shown in FIG. 11, a housing (not shown) for the system 900 can form the base that will result in enclosed flow channels. In another embodiment, the silicon/glass wafer combination described in the previous process may be replaced with a titanium wafer/expansion matched glass combination, but with the titanium patterned using a DRIE process which is similar to that used to pattern silicon but is chlorine based.

The systems described herein can be designed for shunting fluid between a variety of body regions, either alone or in combination with additional components. As noted above, for example, in some embodiments the systems described herein are designed to be implanted in a patient's eye to shunt aqueous between the anterior chamber and a target outflow location (e.g., a subconjunctival bleb space), such as to treat glaucoma. Accordingly, in some embodiments the systems described herein can have dimensions compatible with being implanted in the patient's eye. For example, adjustable shunts incorporating the systems/microvalves described herein (e.g., the system 100) may have a length of between about 4 mm and about 20 mm, such as between about 4 mm and 15 mm, or between about 4 mm and 12 mm, or between about 6 mm and 10 mm, or about 8 mm. In some embodiments, individual components (e.g., layers) can have a width or thickness less than about 500 microns, less than about 400 microns, less than about 300 microns, and/or less than about 200 microns. In some embodiments, the diameter of the fluidic channels and corresponding apertures (e.g., channel 132; channel 232; channel 1138) may be less than about 100 microns, less than about 75 microns, and/or less than about 50 microns, such as about 35 microns. The foregoing dimensions are provided by way of example only, and other dimensions outside the ranges provided above are possible and included within the scope of the present technology. Indeed, the dimensions of the systems described herein may be designed depending on the type of shunting system (e.g., glaucoma shunt vs. hydrocephalus shunt) and intended recipient (e.g., child vs. adult).

Without intending to be bound by theory, one expected advantage of the system 100 is that select components can be manufactured with precision and in large quantities despite the relatively small sizes of these components. For example, the actuator assembly 110 and/or the control assembly 120 can be manufactured as glass and/or silicon wafers before being coupled together to form the system 100. This is expected to increase the efficiency and reproducibility of manufacturing the system 100.

### EXAMPLES

Several aspects of the present technology are set forth in the following examples:

1. An adjustable shunting system for shunting fluid from a first body region to a second body region within a patient, the adjustable shunting system comprising:
an actuator assembly including a shape memory actuator;
a control assembly including a flow control element; and
a fluidics assembly including a lumen having a flow restrictor portion and a diaphragm extending over the flow restrictor portion,
wherein the actuator assembly, the control assembly, and the fluidics assembly are vertically stacked such that the shape memory actuator is positioned within a chamber above the flow control element and the flow control element is positioned above the diaphragm, and
wherein the shape memory actuator is configured to selectively transition the flow control element between at least two positions, and wherein in a first position of the at least two positions, the flow control element deflects the diaphragm into the flow restrictor portion to increase a fluid resistance therethrough.

2. The adjustable shunting system of example 1 wherein the actuator assembly further includes a cover composed at least partially of glass.

3. The adjustable shunting system of example 1 or example 2 wherein the shape memory actuator is composed at least partially of Nitinol.

4. The adjustable shunting system of any one of examples 1-3 wherein the control assembly is composed at least partially of silicon.

5. The adjustable shunting system of any one of examples 1-4 wherein the diaphragm is composed at least partially of glass.

6. The adjustable shunting system of any one of examples 1-5 wherein the chamber comprises an inert gas disposed therein.

7. The adjustable shunting system of example 6 wherein the inert gas comprises argon.

8. The adjustable shunting system of any one of examples 1-7 wherein the system is an intraocular shunting system configured to be implanted within an eye of the patient.

9. The adjustable shunting system of any one of examples 1-8 wherein the actuator assembly, the control assembly, and the fluidics assembly are manufactured as separate components.

10. The adjustable shunting system of any one of examples 1-9 wherein at least a portion of the lumen is serpentine shaped.

11. An adjustable shunting system for shunting fluid from a first body region to a second body region within a patient, the adjustable shunting system comprising:
an actuator assembly including a shape memory actuator;
a first control assembly including a flow control element, wherein the flow control element is movably coupled to the shape memory actuator; and
a second control assembly including a flow control diaphragm, wherein the flow control diaphragm is movably coupled to the flow control element,
wherein, in response to being actuated, the shape memory actuator is configured to rotate the flow control element to change a position of the flow control diaphragm.

12. The adjustable shunting system of example 11 wherein the actuator assembly includes an actuator pin, and wherein the actuator pin movably couples the shape memory actuator and the flow control element.

13. The adjustable shunting system of example 11 or example 12 wherein the second control assembly includes a connector element, and wherein the connector element movably couples the flow control diaphragm and the flow control element.

14. The adjustable shunting system of any one of examples 11-13 wherein the shape memory actuator is disc shaped.

15. The adjustable shunting system of any one of examples 11-14 wherein the shape memory actuator is a first shape memory actuator and the flow control diaphragm is a first flow control diaphragm, and wherein the system further comprises:
a second shape memory actuator, wherein the second shape memory actuator is movably coupled to the flow control element; and
a second flow control diaphragm, wherein the second flow control diaphragm is movably coupled to the flow control element.

16. A microfluidic valve assembly for use with an adjustable shunting system, the microfluidic valve assembly comprising:
a shape memory actuator;
a control assembly having a movable control element and one or more mechanical suspension members; and
a plate having a hole extending at least partially therethrough,
wherein the shape memory actuator is configured to move the movable control element relative to the hole via deflection of the one or more mechanical suspension members.

17. The microfluidic valve assembly of example 16 wherein the shape memory actuator is composed, at least in part, of Nitinol.

18. The microfluidic valve assembly of example 16 or example 17 wherein the control assembly is spaced apart from the plate.

19. The microfluidic valve assembly of any one of examples 16-18 wherein the control assembly has a first surface area, and wherein the hole has a second surface area less than the first surface area such that the control assembly variably obscures the hole as the control element moves over its range of motion.

20. The microfluidic valve assembly of any one of examples 16-19 wherein the control assembly comprises a plurality of mechanical support members arranged in a cantilevered configuration, and wherein the plurality of mechanical support members are sized and shaped to position the control assembly at a desired vertical spacing from the plate.

### Conclusion

The above detailed description of embodiments of the technology is not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the intraocular shunts described herein may be combined with any of the features of the other intraocular shunts described herein and vice versa. Moreover, although steps are presented in a certain order in the embodiments described herein, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense-that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portion(s) of this application. Where the context permits, words in the above Detailed Description using the singular or plural number may also include the plural or singular number, respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An adjustable shunting system for shunting fluid from a first body region to a second body region within a patient, the adjustable shunting system comprising:
an actuator assembly including a shape memory actuator;
a control assembly including a flow control element; and
a fluidics assembly including a lumen having a flow restrictor portion and a diaphragm extending over the flow restrictor portion,
wherein the actuator assembly, the control assembly, and the fluidics assembly are vertically stacked such that the shape memory actuator is positioned within a chamber above the flow control element and the flow control element is positioned above the diaphragm, and
wherein the shape memory actuator is configured to selectively transition the flow control element between at least two positions, and wherein in a first position of the at least two positions, the flow control element deflects the diaphragm into the flow restrictor portion to increase a fluid resistance therethrough.

2. The adjustable shunting system of claim 1 wherein the actuator assembly further includes a cover composed at least partially of glass.

3. The adjustable shunting system of claim 1 wherein the shape memory actuator is composed at least partially of Nitinol.

4. The adjustable shunting system of claim 1 wherein the control assembly is composed at least partially of silicon.

5. The adjustable shunting system of claim 1 wherein the diaphragm is composed at least partially of glass.

6. The adjustable shunting system of claim 1 wherein the chamber comprises an inert gas disposed therein.

7. The adjustable shunting system of claim 6 wherein the inert gas comprises argon.

8. The adjustable shunting system of claim 1 wherein the system is an intraocular shunting system configured to be implanted within an eye of the patient.

9. The adjustable shunting system of claim 1 wherein the actuator assembly, the control assembly, and the fluidics assembly are manufactured as separate components.

10. The adjustable shunting system of claim 1 wherein at least a portion of the lumen is serpentine shaped.

11. An adjustable shunting system for shunting fluid from a first body region to a second body region within a patient, the adjustable shunting system comprising:
an actuator assembly including a shape memory actuator;
a first control assembly including a flow control element, wherein the flow control element is movably coupled to the shape memory actuator; and
a second control assembly including a flow control diaphragm, wherein the flow control diaphragm is movably coupled to the flow control element,
wherein, in response to being actuated, the shape memory actuator is configured to rotate the flow control element to change a position of the flow control diaphragm.

12. The adjustable shunting system of claim 11 wherein the actuator assembly includes an actuator pin, and wherein the actuator pin movably couples the shape memory actuator and the flow control element.

13. The adjustable shunting system of claim 11 wherein the second control assembly includes a connector element, and wherein the connector element movably couples the flow control diaphragm and the flow control element.

14. The adjustable shunting system of claim 11 wherein the shape memory actuator is disc shaped.

15. The adjustable shunting system of claim 11 wherein the shape memory actuator is a first shape memory actuator and the flow control diaphragm is a first flow control diaphragm, and wherein the system further comprises:
a second shape memory actuator, wherein the second shape memory actuator is movably coupled to the flow control element; and
a second flow control diaphragm, wherein the second flow control diaphragm is movably coupled to the flow control element.

16. A microfluidic valve assembly for use with an adjustable shunting system, the microfluidic valve assembly comprising:
a shape memory actuator;
a control assembly having a movable control element and one or more mechanical suspension members; and
a plate having a hole extending at least partially therethrough,
wherein the shape memory actuator is configured to move the movable control element relative to the hole via deflection of the one or more mechanical suspension members.

17. The microfluidic valve assembly of claim 16 wherein the shape memory actuator is composed, at least in part, of Nitinol.

18. The microfluidic valve assembly of claim 16 wherein the control assembly is spaced apart from the plate.

19. The microfluidic valve assembly of claim 16 wherein the control assembly has a first surface area, and wherein the hole has a second surface area less than the first surface area such that the control assembly variably obscures the hole as the control assembly moves over its range of motion.

20. The microfluidic valve assembly of claim 16 wherein the control assembly comprises a plurality of mechanical support members arranged in a cantilevered configuration, and wherein the plurality of mechanical support members are sized and shaped to position the control assembly at a desired vertical spacing from the plate.
